(19) United States
(12) Patent Application Publication
(10) Pub. No.: US 2016/0114985 A1
(43) Pub. Date: Apr. 28, 2016

(54) Needle Bevel Orienting Device

(71) Applicants: Thomas J. Shaw, Frisco, TX (US); Retractable Technologies, Inc., Little Elm, TX (US)

(72) Inventors: Thomas J. Shaw, Frisco, TX (US); Steve R. Wisner, Frisco, TX (US); Mark Small, Heavener, OK (US)

(21) Appl. No.: 14/726,210

(22) Filed: May 29, 2015

Related U.S. Application Data
(60) Provisional application No. 62/004,669, filed on May 29, 2014.

(51) Int. Cl. B65G 47/24

(52) U.S. Cl. CPC B65G 47/24; A61B 2017/00526

(57) ABSTRACT

An apparatus and method for selectively orienting beveled needle tips by use of cooperatively configured, counter-rotating curved surfaces that contact and rotate the needle to a preferred angular orientation without blunting or marring the bevel edges. The counter-rotating surfaces may be selectively repositioned at different angles in order to reliably orient all needles as part of a multi-step method.

### FIELD OF THE INVENTION

This invention relates to the manufacture of medical products and devices having needles with beveled tips, particularly the manufacture of medical products in which the needles have beveled tips that are desirably oriented in a preferred orientation relative to another portion of the associated product.

### DESCRIPTION OF RELATED ART

Needles for medical devices are typically made of medical grade stainless steel and have a beveled section disposed at the projecting free end of the needle. When such a needle is assembled into a medical device, it is often preferred that the beveled section be oriented in a desired rotational alignment to another part of the device, particularly where the needle is intended for vascular insertion. U.S. Pat. No. 4,436,479 discloses a device for orienting intravenous needles.

A device is needed that can quickly and reliably orient needle bevels without damaging the bevel edges. Known needle alignment methods are typically capable of orienting large numbers of needles quickly with an acceptable failure rate, or of orienting a few needles slowly but with greater certainty. However, high production rates and high quality and reliability without damaging needle bevels are both desired in modern large-scale manufacturing environments.

### SUMMARY OF THE INVENTION

A device and method of use are disclosed that will quickly and reliably align a needle bevel in a preferred angular orientation to another part of a medical product during a high speed manufacturing process without damaging the edges of the needle bevels.

In one embodiment of the needle, apparatus is disclosed comprising a frame having a plurality of roller heads that are each pivotably mounted to facilitate sequentially contacting a needle bevel from each of two different angular positions if needed for reasons discussed below. Similarly, a plurality of needles are desirably supported by needle holders disposed in another device (not shown) in a substantially vertical position relative to the frame, and are desirably indexed in a desired position relative to the frame with a needle bevel facing upwardly in substantial vertical alignment with each roller head. Each roller head desirably has a pair of needle-orienting rollers and is configured to be lowered when a needle is positioned beneath it for rotating the needle around its longitudinal axis to orient the bevel in a desired rotational position relative to the needle holder.

In one embodiment of the invention of the invention, each roller head comprises a pair of counter-rotating rollers that cooperate to contact and rotate a needle until the needle bevel is disposed in a desired rotational position. Each roller head desirably has a longitudinal axis that lies in a plane substantially transverse to the plane of the longitudinal axis through the associated needle. (Because each needle will likely have some initial degree of “wobble” with respect to its supporting needle holder due to a slight clearance between the inside diameter of the needle holder and the outside diameter of the needle, the needle may not be perpendicular prior to engagement with the rollers.) The rollers, preferably cylinders, each comprise a curved outer surface, and the curved surfaces are oriented so that their axes of rotation are parallel and the surfaces are separated by a gap that is slightly less than the outside diameter of the needle to be oriented. The rollers are desirably supported by a frame member and are configured and installed to rotate in opposite directions at rotational speeds that provide the same linear rate of advance relative to a needle disposed between them. In one embodiment of the invention, the radius of curvature of the curved outer surface of the rollers used to orient the needle bevel are desirably equal and significantly greater than the radius of curvature of the outside surface of the needle (eg., 4-10 times the radius of curvature of the needle). Alternatively, each of the opposed curved surfaces can have a different radius of curvature if their respective rates of rotation are controlled so as to provide substantially the same linear rate of advance in relative to a needle disposed between them.

In one embodiment of the invention, the counter-rotating rollers are lowered from above the needle, which is desirably supported by a needle holder in a vertical position that is cooperatively aligned with the gap space between the rollers. The rollers desirably descend at a rate that substantially matches the linear surface speed of the rollers so that the relative speed between the needle tip and the roller surfaces is effectively zero when they make contact. This rate matching allows the needle tip to be received into the gap between the rollers without sliding, scratching or grinding at the opposed points of contact between the rollers and the needle that could otherwise damage the needle bevel.

As the opposed roller surfaces contact the needle and continue downwardly, and depending upon the angular relationship of the bevel to the contacting roller surfaces at the time of initial contact, the beveled needle tip is desirably pressured by the opposed roller surfaces so that the needle is rotated around its longitudinal axis until the bevel disposed in a preferred orientation (sometimes referred to as a 0° orientation) relative to the needle holder or to another reference objective.

A single application of the needle orienting device of the invention is usually sufficient for properly orienting a needle bevel regardless of the initial orientation, and most starting needle bevel orientations less than + or −90° can be successfully corrected to achieve a 0° orientation with a single application. However, where the initial needle bevel orientation is very close to 90° and the rotational force component applied to each side of the needle bevel is substantially the same, the needle may resist rotational movement and be “blocked” or “wedged” between the rollers without rotation. To account for such an occurrence, the needle orienting device of the invention is preferably configured to be adaptable for use in a two-step process that will produce a different relative rotational alignment between the counter-rotating roller surfaces and the needle to produce the desired rotational movement of the needle to a 0° orientation. This can be achieved in many different ways.

To deal with instances where the initial rotational alignment of the needle prevents rotation, one can modify either the needle orienting device or the needle positioning apparatus to produce a different initial rotational alignment between the needle and the counter-rotating roller surfaces. This can be done, for example, by configuring the roller support frame to allow pivoting of the parallel rotational axes of the roller surfaces and the gap between the rollers relative to the needle, which can be achieved by raising the supporting frame member and then lowering it again after the frame member has been pivoted to another position. Alternatively, or in combination with this adaptation, the needle positioning apparatus can be rotated to a different angular position relative to the counter-rotating roller surfaces, or the needle can be rotated slightly within the needle positioning apparatus to another rotational position that will allow the rollers to reposition the needle bevel to a desired orientation.

In order to achieve a large production volume of needles, the preceding mechanism and principles can be easily scaled up from a single roller pair and needle to multiple roller pairs and needles. A linear array of roller surfaces having parallel rotational axes can be configured to pivot about independent axes while maintaining mutual parallelism. It is intended that this pivoting and the resulting lateral displacement of the rollers be calibrated so that each roller pair is capable of being repositioned over an adjacent needle in an array of needles. With this configuration, each needle can be oriented by two sets of roller pairs, each at differing angles relative to the needle, thereby allowing for the described multi-step process if necessary. The number of roller heads is desirably one greater than the number of needles in any one batch to accommodate adjusting each roller head from a first angular position to a second angular position relative to the needle bevel during the bevel orientation process.

In one embodiment of the invention, after each needle bevel in a group of needles has been oriented by the subject apparatus to a desired rotational position relative to the needle holder supporting the needle, each needle is desirably fixed in that desired rotational position by some satisfactory method such as applying a drop of glue and spot curing it by the use of conventional means. Fixing the needle in the proper orientation is desirable to prevent the needle from again rotating due to vibration or other factors after the roller heads are raised the second time to disengage them from the needle bevel.

### BRIEF DESCRIPTION OF THE DRAWING

The apparatus of the invention is further described and explained in relation to the following drawings wherein:

FIG. 1 is a front elevation view of a roller head, its constituent components, and a rack rod;

FIG. 2 is a bottom perspective view of the roller head of FIG. 1; FIG. 3 is a simplified diagrammatic, front elevation view of a needle at an initial 60° orientation in relation two counter-rotating rollers;

FIG. 4 is a composite view of a front elevation view of a needle at a final 0° orientation in relation to two counter-rotating rollers;

FIG. 5 is a composite view of a front elevation view of a needle at an initial 90° orientation in relation to two counter-rotating rollers;

FIG. 6 is a perspective view of a preferred embodiment of the apparatus;

FIG. 7 is a side elevation view of the apparatus of FIG. 6;

FIG. 8 is a top perspective view of the apparatus of FIG. 7;

FIG. 9 is a rear perspective view of the apparatus of FIG. 7;

FIG. 10 is front perspective view of another embodiment of the apparatus of the invention that depicts a plurality of roller heads, with at least one roller head being pivoted to an alternate position disposed at a different angle of rotation in relation to the other roller heads;

FIG. 11 is a top front perspective view of one simplified embodiment of a needle support assembly suitable for use with the apparatus of the invention; and

FIG. 12 is a rear perspective view of the apparatus of FIG. 6-9.

### DESCRIPTION OF THE PREFERRED EMBODIMENTS

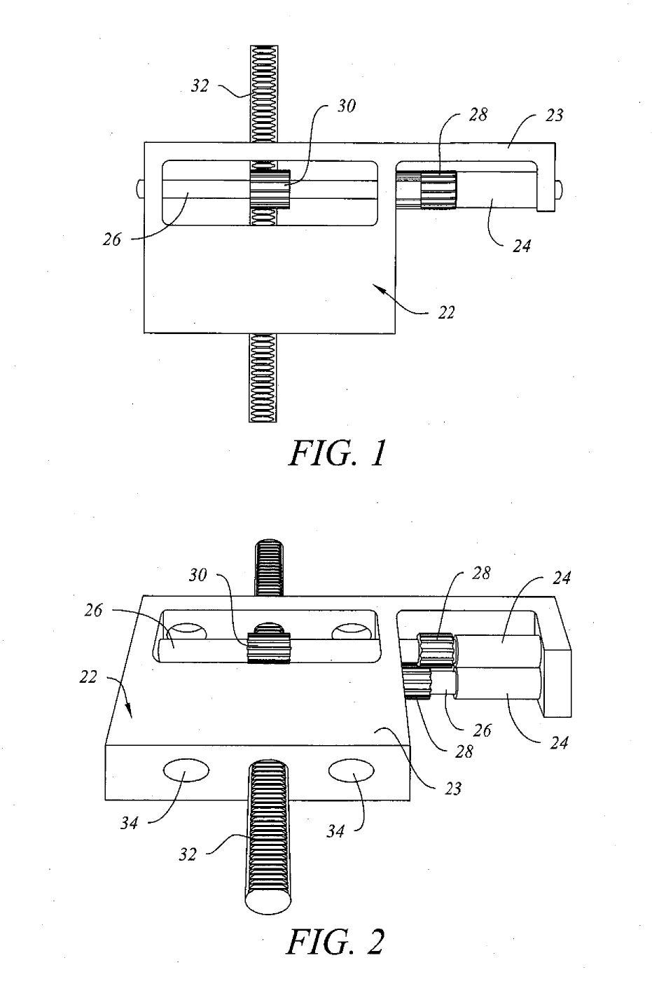

As shown in FIGS. 1 and 2, roller head 22 comprises rollers 24, spindles 26, idler gears 28, drive gear 30, and housing body 23. The rollers 24 are cylinders which are fixed to spindles 26. These spindles 26 each additionally support idler gears 28, idler gears 28 being meshed together so that the rotational rates of rollers 24 are set to a fixed ratio. This ratio is defined to be that which causes the linear speed of the roller surfaces to be equal to each other. Preferably such a ratio would be 1:1 so that rollers 24 would be of equal diameter, but this is not required. Idler gears 28 enable rollers 24 to rotate in opposite directions, as indicated by arrows 82, 84 in FIGS. 3-5. Another third gear, drive gear 30, is disposed on one of spindles 26 and interfaces with rack rod 32. The relative motion between this drive gear 30 and rack rod 32 drives the rotation of one spindle 26 as well as roller 24 and idler gear 28 on this driven spindle. Due to the intermeshing between idler gears 28, the other idler gear 28, spindle 26, and roller 24 are compelled to rotate as well. A further embodiment would be to eliminate idler gear 28 which shares spindle 26 with drive gear 30, and relocate the remaining idler gear 28 so that it meshes with drive gear 30 in order to provide power to roller 24 which does not share spindle 26 with drive gear 30.

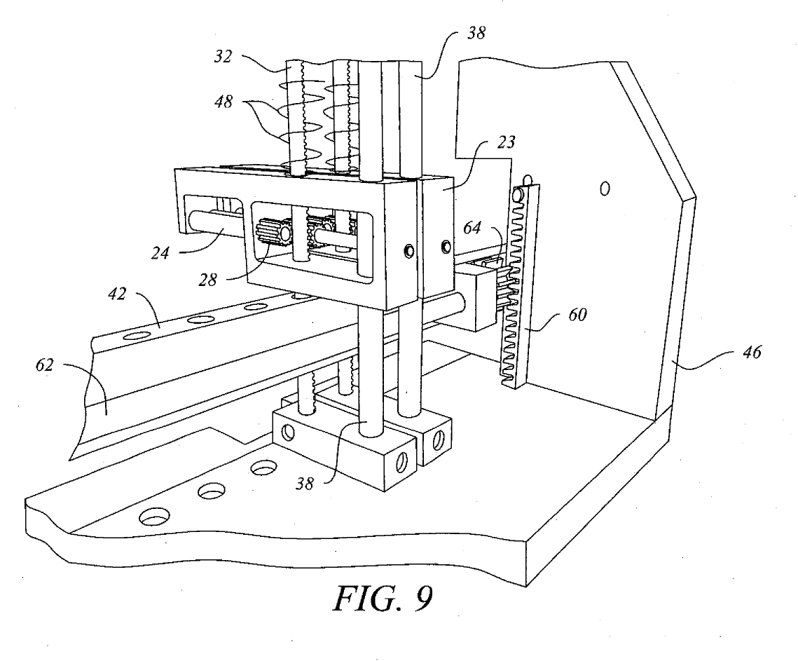

All of the aforementioned components are disposed within roller head 22, which is a structure comprising body 23 that provides a bearing surface for spindles 26 and contains rollers 24, idler gears 28, and drive gear 30. Additionally, pivot rod apertures 34 are provided through body 23 to receive pivot rod 38d (FIG. 9) which controls the yawing motion of roller head 22. Drive gear 30 is sized so that the linear speed of the outer surface of rollers 24 relative to roller head 22 will equal the speed of roller head 22 as it moves up and down rack rod 32. This arrangement in intended to avoid relative motion between needle 25 (FIGS. 3-5) and the outer surface of rollers 24 as roller head 22 lowers rollers 24 into position to engage needle 25.

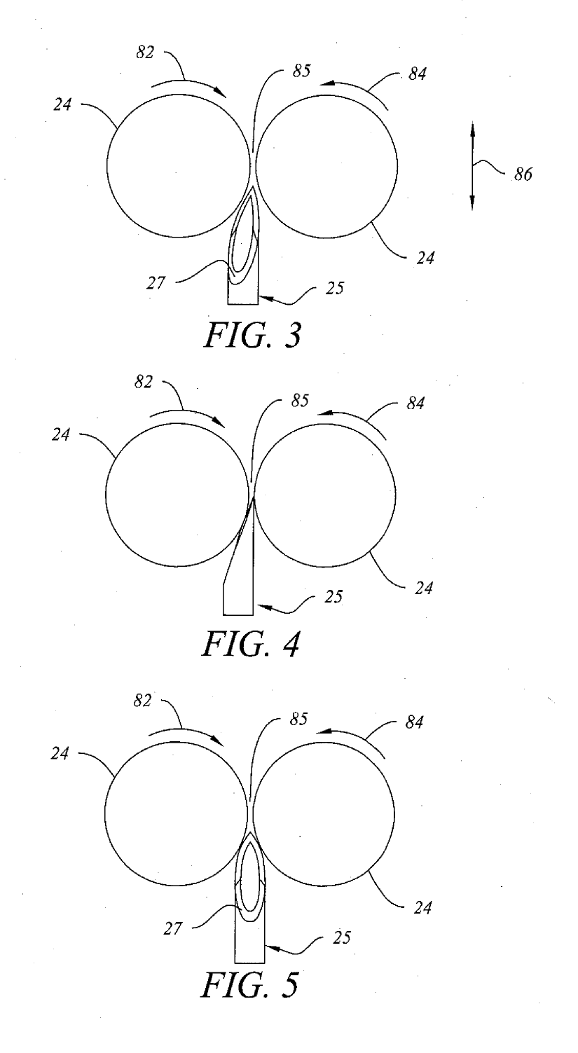

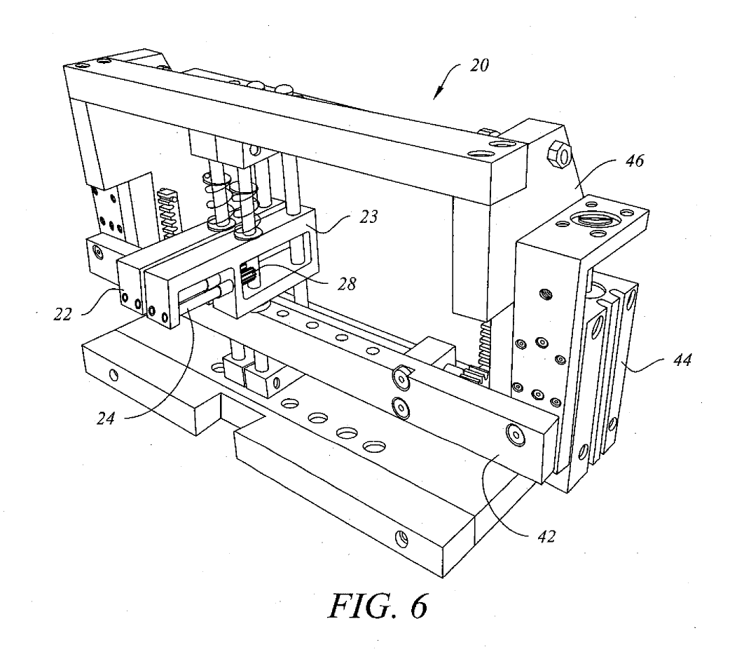

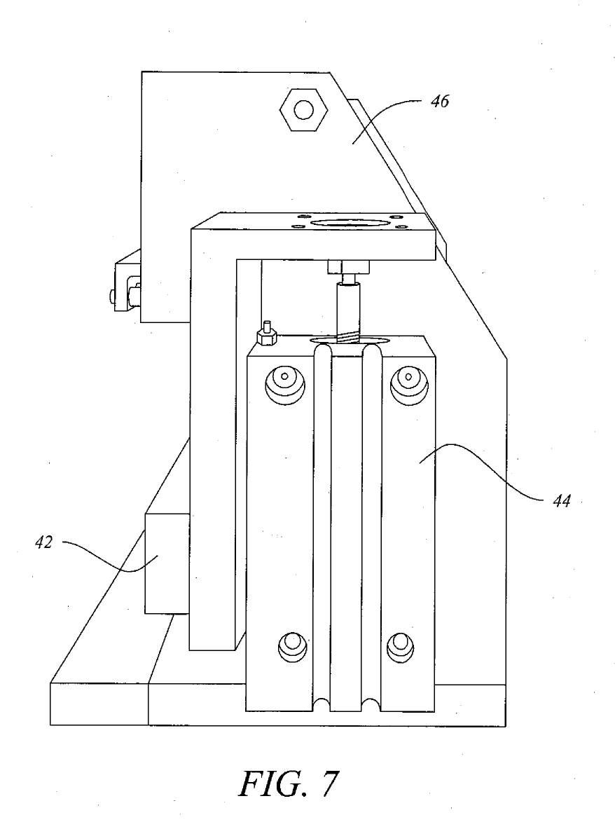

FIGS. 3-5 illustrate three of an infinite number of different orientations that needle 25 may assume relative to rollers 24 during use of the disclosed method. FIG. 3 shows a typical orientation, one that is not too close to either extreme of 0° as in FIG. 4, or 90° as in FIG. 5. Needle 25 in FIG. 3 is shown in its un-oriented state, prior to application of the method. Once rollers 24 descend into contacting relationship to needle 25, bevel 27 will be reoriented to the final, preferred state of 0° as seen in FIG. 4. If needle 25 were to begin in the 0° configuration, rollers 24 would have no effect when lowered. Likewise, rollers 24 are sometimes ineffective in reorienting needle 25 when it is at the 90° or near-90° orientation as in FIG. 5. This limitation is what makes a two-step approach to the orientation of needle 25 desirable. Using a two-step method, if needle 25 is initially oriented in a 90° or near-90° position, roller head 22 is desirably pivotable relative to frame 46 (FIGS. 6-9) to reposition rollers 24 to another angular relationship with needle 25 that will allow rollers 24 to orient bevel 27 (FIGS. 3) of needle 25 to a desired final position. It should be appreciated by those of skill in the art upon reading this disclosure in relation to the accompanying drawings that FIGS. 3-5 are simplified diagrammatic views that are not drawn to scale, meaning for example that both the ratio of the radius of curvature of rollers 24 to that of needle 25 and the separation between rollers 24 in gap 85 will desirably be relatively greater than shown in FIGS. 3-5. Gap 85 will desirably be larger than shown (by comparison) but slightly smaller than the outside diameter of needle 25 to facilitate engagement with needle 25 at a point lower on bevel 27 than shown to rotate needle 25 more effectively and to accommodate some degree of “wobble” of needle 25 from a strictly vertical position as it is supported in the needle holder prior to repositioning bevel 27.

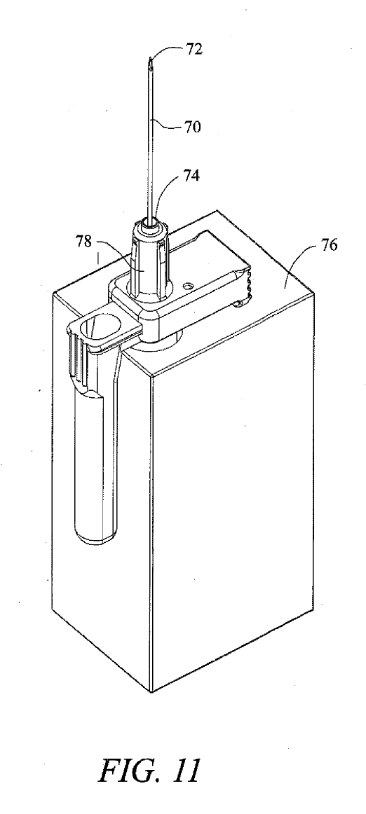

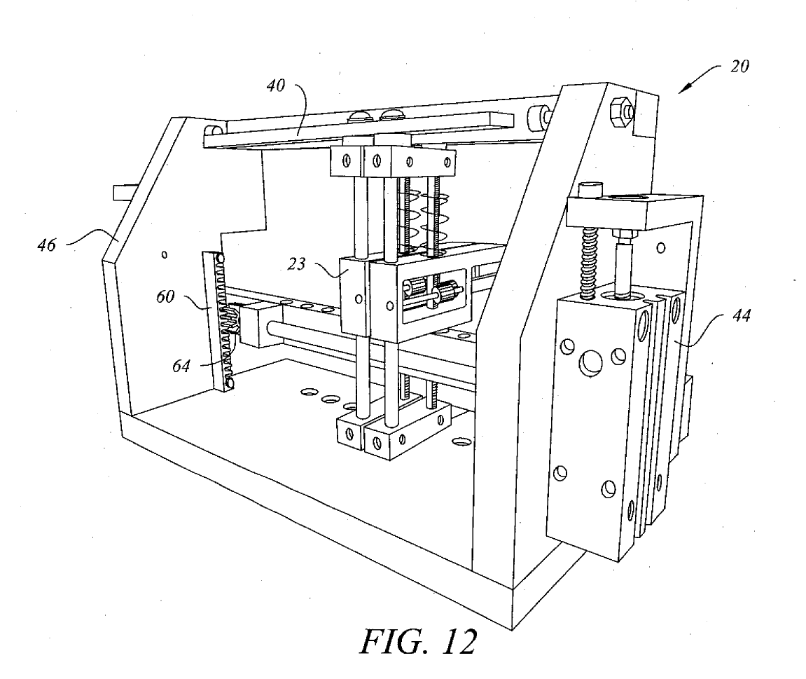

Apparatus 20 for multiple simultaneous needle orientations is presented in FIG. 6. Two roller heads 22 are displayed for simplicity, but apparatus 20 is intended to support many more roller heads 22 in a similar configuration. In FIG. 6, needle positioning bar 60 attached to frame 46 and having a single indexing notch 62 is provided to facilitate test placement of a single needle/needle holder/support assembly beneath a roller head 22. One such single needle/needle holder/support assembly suitable for test use is described below in relation to FIG. 11. Roller heads 22 are positioned in the apparatus by way of their rack rods 32. Each rack rod is fixed to frame 46 and provides an axis about which roller head 22 may pivot. Roller heads 22 are supported vertically by lift bar 42 which is motivated by actuator 44. Lift bar 42 raises roller heads 22 all at once, and once needles 25 are in their positions below rollers 24, lift bar 42 is lowered, allowing roller heads 22 to descend under their own weight. Springs 48 (FIG. 9) provide additional downward force to assist rollers 24 in orienting needle bevels 27 (FIGS. 3, 5). The upward and downward motion of rollers 24 relative to needle 25 within a predetermined range of travel is depicted in simplified diagrammatic form by arrow 86 in FIG. 3.

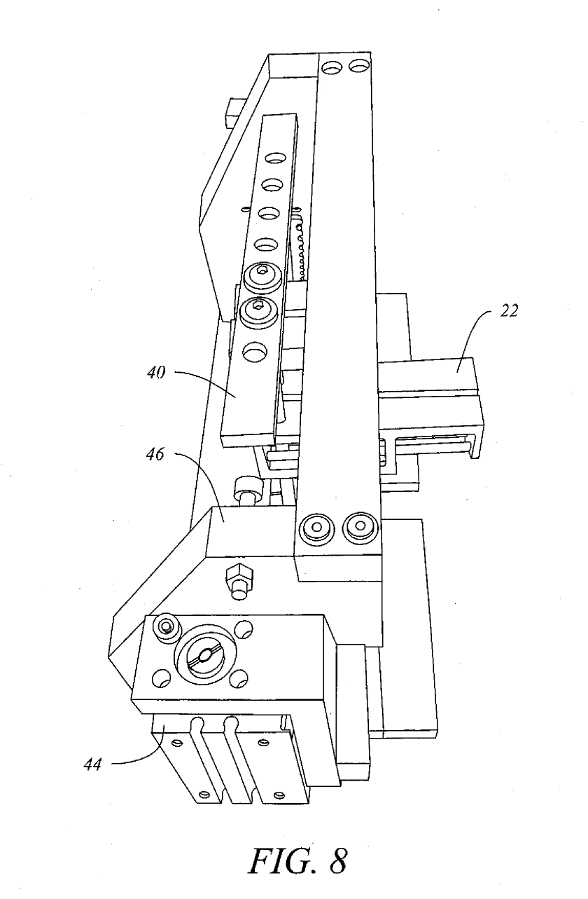

FIG. 9 allows for a closer view of roller heads 22 and their related components when installed. As described previously, roller heads 22 are laterally fixed about their rack rods 32, and supported by lift bar 42. Pivot rods 38 are disposed through pivot rod apertures 34 in the end of body 23 opposite rollers 24, These pivot rods 38 are all connected to pivot bar 40 as seen in FIG. 8. This pivot bar 40 selectively controls the angle of roller heads 22 by translating parallel to its own long axis, thereby moving pivot rods 38 and forcing roller heads 22 to pivot about their rack rod 32.

Starting with an already-raised array of roller heads 22, pivot bar 40 is desirably moved so that roller heads 22 are pivoted to another angle with respect to frame 46. This positions an array of needles (not shown except in FIGS. 3-5) below roller heads 22 so that each needle 25 is aligned with the space between rollers 24, retracting actuator 44 which lowers lift bar 42 and roller heads 22, extending actuator 44 to raise the bar 42 and roller heads 22, translating pivot bar 40 so that roller heads 22 are perpendicular to the pivot bar's long axis, retracting actuator 44 which lowers lift bar 42 and roller heads 22 onto needles 25, extending actuator 44 to raise lift bar 42 and roller heads 22, and removing the array of oriented needles 25.

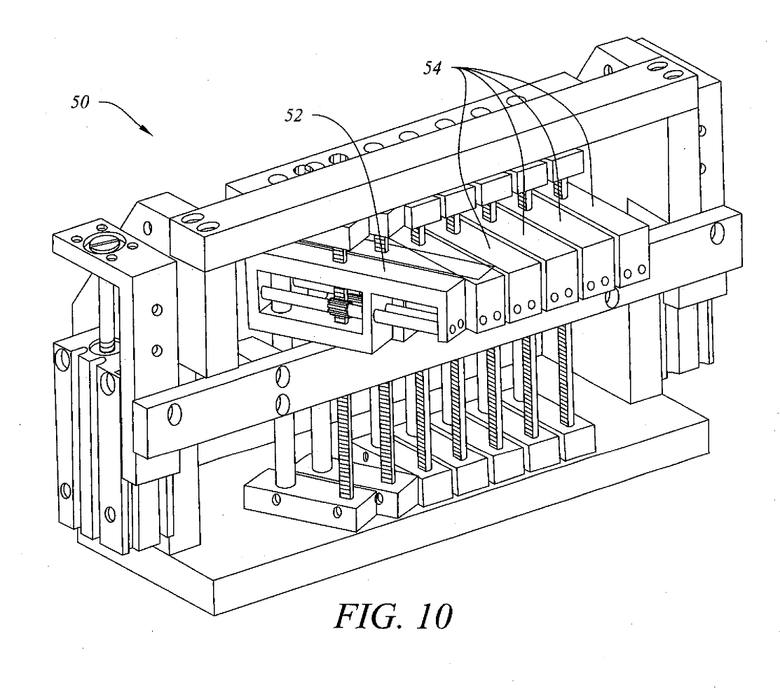

FIG. 10 depicts another embodiment of apparatus 50 of the invention in which the pivotal movement of roller head 52 relative to other roller heads 54 is further depicted.

FIG. 11 depicts a needle 70 having projecting needle bevel 72. Needle 70 is initially rotatably disposed in relation to and is supported in a substantially vertical orientation by needle holder 74. Needle 70 and needle holder 74 are maintained in a desired positional relationship to apparatus 20 of the invention by needle support member 76 of a type that is suitable for use in apparatus 20 of the invention as configured and previously described in relation to FIG. 6, More particularly, in the embodiment shown in FIG. 11, needle support member 76 is configured as a test piece to fit inside indexing notch 62 in positioning bar 60. As previously mentioned, needle 70 may be able to “wobble” to some extent inside needle holder 74 because rotational movement of needle 70 within needle holder 74 is desirably permitted to allow the orientation of needle bevel 72 to be repositioned relative to needle holder 74, after which needle 70 can be spot glued to needle holder 74 to prevent further rotational movement of needle 70 relative to needle holder 74. Alternatively, needle 70 can be set in a fixed position relative to needle holder 74 if needle holder 74 is rotatable relative to needle assembly 78 in such manner that their relative rotational positions can be fixed prior to properly orienting needle bevel 72.

Other alterations and modifications of the subject invention will likewise become apparent to those of ordinary skill in the art upon reading this disclosure in relation to the accompanying figures of the drawings, and it is intended by the inventors that the scope of the invention as disclosed here be limited only by the broadest possible interpretation of the association claims to which the inventors are legally entitled.

What is claimed is:

1. A device for orienting a needle bevel relative to a needle support member, the device comprising a pair of rollers that cooperate to contact and rotate a needle about its longitudinal axis until the needle bevel is repositioned to a desired rotational alignment with the needle holder.

2. The device of claim 1 wherein the each of the rollers comprises a curved outer surface, and the curved surfaces are oriented so that their axes of rotation are parallel and the surfaces are separated by a gap that is slightly less than an outside diameter of the needle to be oriented.

3. The device of claim 2 wherein the rollers are desirably supported by a frame member and are configured and installed to rotate in opposite directions with outer surfaces rotating at rotational speeds that provide the same linear rate of advance relative to a needle disposed between them.

4. The device of claim 3 wherein the frame member supporting the rollers is aligned and configured to move from a first position where the needle bevel is aligned with the gap to a second position where each roller is contacting the needle and at least one roller is contacting an edge of the needle bevel.

5. The device of claim 4 wherein the frame member moves from the first position to the second position at a linear rate of travel that is equal to the linear rate of advance of each outer roller surface.

6. The device of claim 3 wherein the frame member is configured to be pivoted between a first needle engagement position and a second needle engagement position.

7. A method for rotating a supported needle relative to a needle support member to reposition a bevel disposed on a free end of the needle relative to the needle support member by selectively contacting the needle with a pair of oppositely disposed, laterally spaced apart, counter-rotating, substantially cylindrical surfaces.

8. The method of claim 7 wherein the needle is contacted by opposed, counter-rotating surfaces having rotational axes parallel to each other and perpendicular to a centrally disposed longitudinal axis of the needle.

9. A method for orienting the bevel portion of a needle to a desired position relative to the needle holder comprising sequentially contacting at least one surface of a portion of the needle from at least two different rotational angles.
